(19) United States
(12) Patent Application Publication
(10) Pub. No.: US 2006/0270024 A1
(43) Pub. Date: Nov. 30, 2006

(54) Bioreactor

(75) Inventor: Christian Uphoff, Aschau i. Chmg. (DE)

Correspondence Address: OLIFF & BERRIDGE, PLC, P.O. BOX 19928, ALEXANDRIA, VA, 22320, US

(73) Assignee: Georg Fritzmeier GmbH & Co. KG, Grosshelfendorf (DE)

(21) Appl. No.: 10/563,372

(22) PCT Filed: Jul. 8, 2004
(86) PCT No.: PCT/DE04/01491

§ 371 Date: May 9, 2006

(30) Foreign Application Priority Data
Jul. 8, 2003 (DE) 103 30 959.4
Jul. 8, 2003 (DE) 103 61 996.8

(51) Int. Cl. C12M 1/00

(52) U.S. Cl. 435/292.1

(57) ABSTRACT

What is disclosed is a bioreactor comprising a strainer basket, inside which a filler body consisting of a porous carrier having a high specific surface area is received. Into this strainer basket a mixture of microorganisms is introduced which preferably includes a proportion of photosynthetically active microorganisms and a proportion of light-emitting microorganisms, so that a photodynamic decomposition of organic substances takes place. In accordance with the invention, the mixture of microorganisms contains a proportion of photocatalytically active nanoparticles.

The invention concerns a bioreactor in accordance with the preamble of claim 1, a microbiotic mixture suited for such a bioreactor, as well as a retrofit kit for a small-scale sewage treatment plant, which is executed with such a bioreactor.

When a township or community is not in a position of constructing a separate connection to a collective sewage drain for a real-estate proprietor, the latter as a rule has to construct a small-scale sewage treatment plant if the duty of effluent disposal was transferred to him. Such small-scale sewage treatment plants are included within the piece of land in question and generally serve for the treatment of the domestic effluent. Having passed through the small-scale sewage treatment plant, the treated effluent is either allowed to seep away—where the ground is capable of absorbing it—or conducted to the nearest open body of water.

For a mechanical purification of the effluent, multi-chamber settling tanks are frequently used in which the undissolved substances are removed from the effluent by settling towards the bottom or by floating to the surface. Multi-chamber settling tanks may, for instance, be constructed as two- or three-chamber tanks, with these chambers being formed in a common receptable and connected with each other such that the water may flow through the chambers without the settled or floated, undissolved substances.

In particular older houses and pieces of land are frequently provided with such multi-chamber settling tanks, the purification capacity of which does, however, as a general rule not satisfy the legislator's provisions. Owing to the high investment costs for the construction of a new small-scale sewage treatment plant including a mechanical and a biological separating stage, it is frequently preferred to retrofit the existing multi-chamber plants with a biological stage.

Reliable decomposition of organic pollutants in the effluent, waste air, or in solids, such as contaminated structures, in the pore system of which oil residues caused by leaked heating oil had collected during past inundations, is an essential demand to modern processing plants.

In documents DE 100 62 812 A1 and DE 101 49 447 A1 it is proposed to decompose these undesirable organic constituents in fluids and solids by means of a microbiotic mixture which contains a proportion of photosynthetically active microorganisms and a proportion of light-emitting microorganisms. This mixed culture was employed with great success in the purification of communal and industrial effluent as well as in the sanitation of structures contaminated-with oil residues.

In post-published patent application DE 102 53 334 a further development of the microbiotic mixed culture is achieved by modifying the latter such that photosensitizers are incorporated into the cells of the organic pollutants during the decomposition process, and then, by means of stimulating these photosensitizers with light, singlet oxygen or other radicals are formed which accelerate the decomposition of the organic constituents.

It was found, however that in particular applications these microbiotic mixed cultures do not unfold the effectiveness required for a reliable decomposition of the organic constituents.

In contrast, the invention is based on the object of furnishing a bioreactor enabling a reliable decomposition of organic pollutants in fluids at a simple structure in terms of apparatus technology. The invention moreover has the purpose of furnishing a microbiotic mixed culture adapted to be used in such a bioreactor.

This object is achieved, in regard of the bioreactor, through the combination of features claim 1, in regard of the microbiotic mixed culture through the features of independent claim 19 and through a retrofit kit for purification plants having the features of claim 24.

In accordance with the invention a bioreactor is being proposed which comprises a container with recesses through which the effluent freighted with organic matter may pass. Inside the container a filler body, hereinafter also referred to as a carrier, is arranged which is designed with a comparatively large specific surface area, so that a large substance exchange surface for the digestion and conversion of the biological constituents of the effluent is available. In accordance with the invention, microorganisms for the decomposition of these organic components are moreover provided inside the container. These microorganisms adhere as a biofilm in the pore system of the porous carrier, so that owing to the effective substance exchange surface an extremely efficient biological conversion is made possible.

This carrier is advantageously introduced spirally into the container, with rotatable mounting of either the carrier relative to the container, or of the latter relative to the carrier. By means of a suitable flow management and/or coating—which will be discussed further below—of the container and owing to the spiral-shaped construction of the carrier, the latter or the entire container may be made to rotate, so that mixing is improved and the biological conversion is increased in comparison with conventional constructions.

The carrier may either be formed by a material executed with a pore system that is applied on a supporting layer, or on the other hand the material having a large specific surface area, which possible is mechanically not very stable, may be introduced between a stable, recessed double wall whereby the mechanical strength of the carrier is determined. In principle it is also possible to execute the carrier of a porous material, for instance a ceramic material, having a large specifc surface area.

In a preferred practical example of the invention, the porous carrier is constituted by a foam material, for instance polyurethane foam, which is covered with a material that is catalytically active and/or provides a large sorption area, such as activated carbon or charcoal.

In accordance with a practical example of the invention it is preferred if a major surface of the preferably spiral-shaped carrier is coated with a material favoring formation of a biofilm, e.g., activated charcoal, and the other major surface with a carrier substance containing the microbiotic mixture. In this structure on the one hand a biofilm forms, while on the other hand the formation of a biofilm on the layer having the added microorganisms is prevented by catalytic activities.

The microorganisms required for the biological conversion are either adhered in advance in the pore system of the carrier by suitable process management, or they are continuously supplied to the process.

In a preferred practical example of the invention, the photocatalytic layer is applied both on the inner circumferential surface and on the outer circumferential surface of the container. Here it is particularly preferred if the photocatalytic layer is applied on the outer circumferential surface in the form of stripes, wherein these stripes may extend in the longitudinal direction of the bioreactor—i.e., in the case of a cylindrical bioreactor these stripes extend in parallel with the longitudinal axis.

The recesses of the container are preferably formed by punching, with the punching burrs extending inwardly, into the enclosed inner space of the bioreactor. By means of these comparatively sharp punching burrs, faults in the coating are formed on which a biofilm preferably forms in operation.

The efficiency of the bioreactor may be enhanced further if a photocatalytic layer, e.g. of titanium dioxide or indium-tin oxide, is at least partially applied on the container walls and/or on the carrier.

The container may be executed in the shape of a cylinder with an end face open from below, or in a funnel shape. In the latter case, the side walls of the container tapering downwardly are provided with recesses for the effluent, while the lower end face is closed. I.e., in the latter case a flow through it takes places approximately in a radial direction, whereas in the former case a flow through it takes places in an axial direction from bottom to top.

For its use in a purification plant, the bioreactor is provided with an amount of buoyancy so that it will float in the chamber, e.g., of a multi-chamber tank. Here it is preferred if the strainer basket is slidably guided in the vertical direction, so that an adaptation to a varying fluid level is made possible.

As was already mentioned, the microorganisms may be introduced into the carrier material. In a preferred solution, the microorganisms are bound in quitosane or a biopolymer, and the carrier, preferably the PU foam coated with activated charcoal, is impregnated with this mixture.

The microbiotic mixture in accordance with the invention moreover contains—in addition to the light-emitting and photosynthetically active microorganisms—a proportion of nano-composite materials, including a preferably piezoelectric core, the surface of which is provided with a photocatalytically active layer.

This nano-composite material has in a preferred practical example a fiber-type structure with a length of 20 to 100 nm and a diameter of 2 to 10 nm.

The photocatalytically active coating is provided with multiple recesses for the formation of pole sites. In the above described fiber-type structures, the poles are formed on the end sides.

The bioreactor in accordance with the invention may be used with minimum complexity for retrofitting a small-scale sewage treatment plant, however may also be used independently as a stage of a processing plant.

Further advantageous developments of the invention are subject matter of further subclaims.

In the following, preferred practical examples of the invention shall be explained in more detail by referring to schematic drawings, wherein:

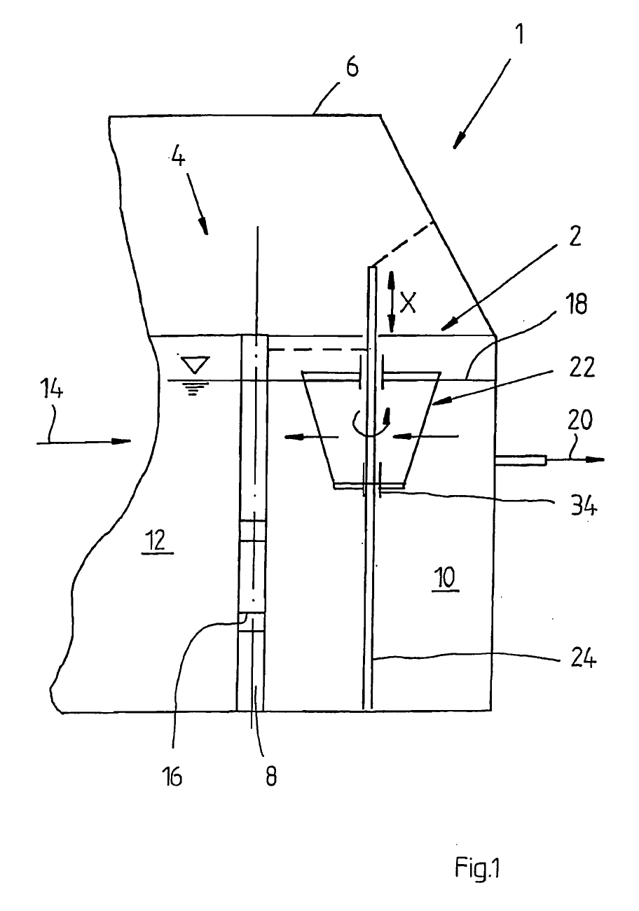

FIG. 1 is a schematic representation of a multi-chamber tank including a retrofitted biological stage;

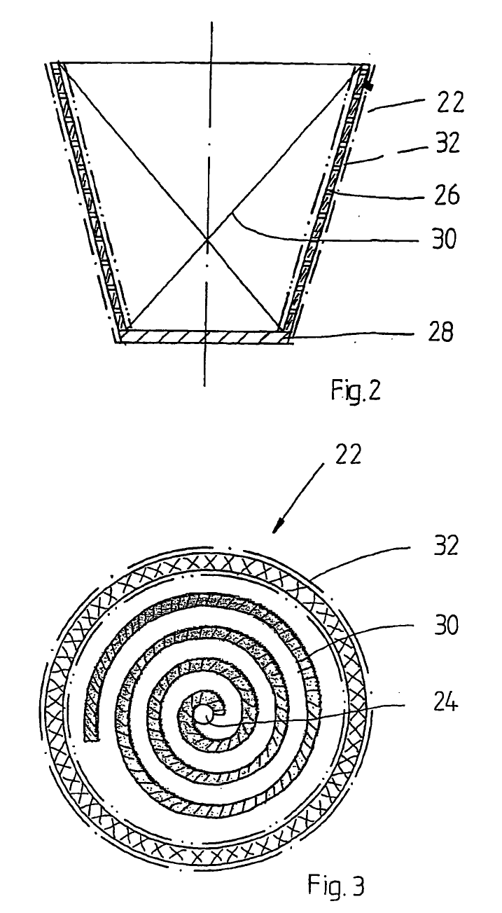

FIG. 2 shows a bioreactor of the biological stage in accordance with FIG. 1;

FIG. 3 is a sectional view of the bioreactor of FIG. 2;

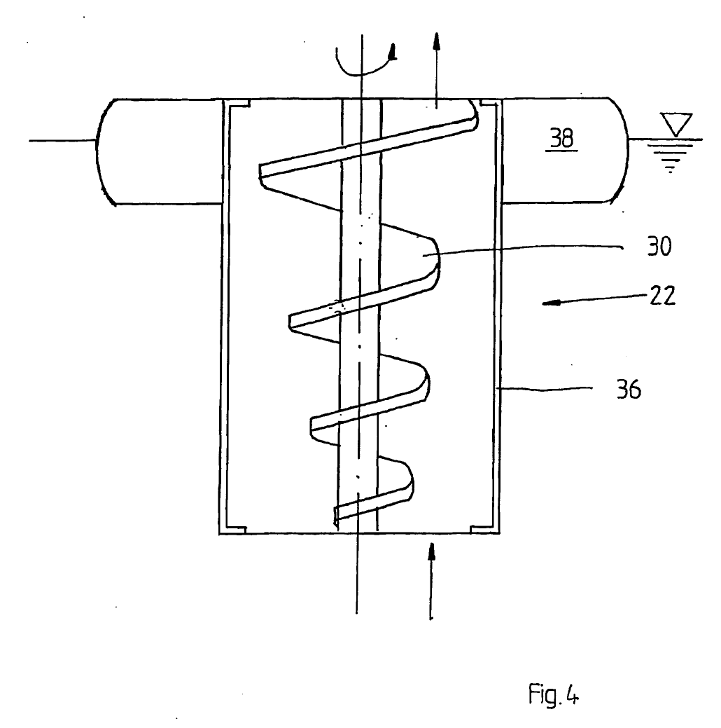

FIG. 4 is a schematic representation of another practical example of a bioreactor for a retrofitted small-scale sewage treatment plant in accordance with FIG. 1;

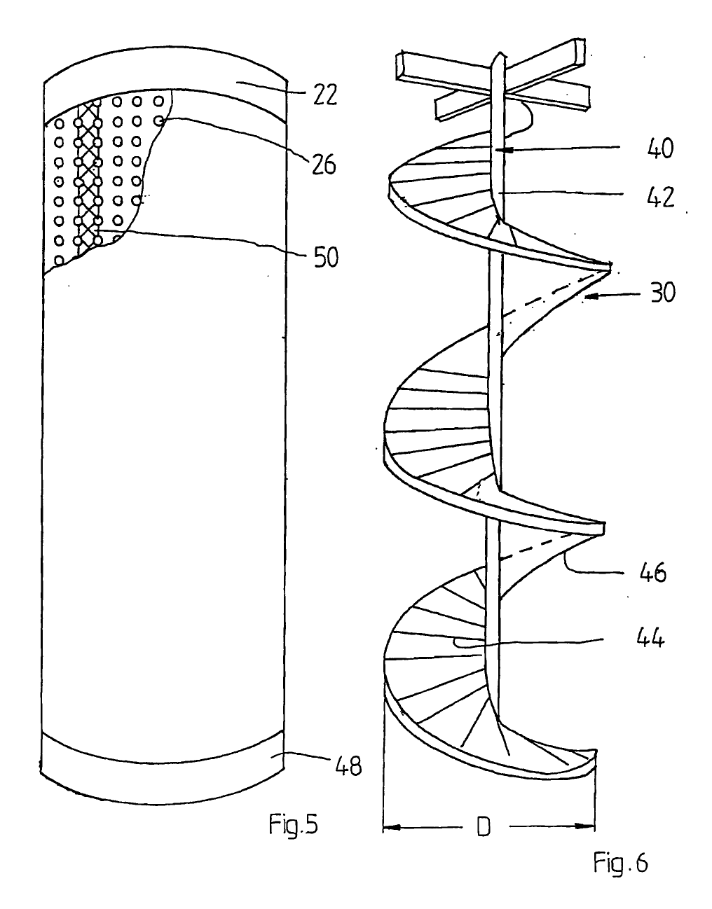

FIG. 5 is a representation of another practical example of a cylindrical bioreactor;

FIG. 6 is a view of a filler body of the bioreactor of FIG. 5;

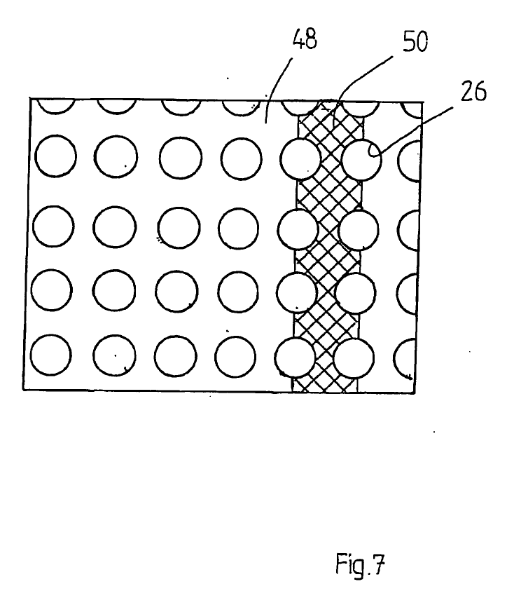

FIG. 7 is a detail representation of the wall of a strainer vessel of the bioreactor of FIG. 5;

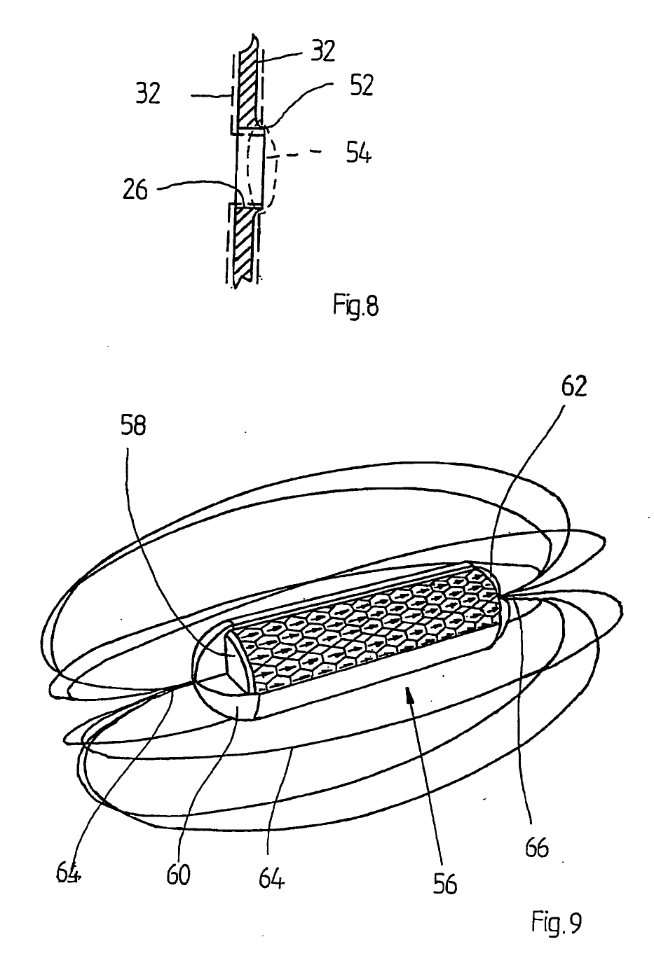

FIG. 8 is a sectional view of the wall of FIG. 8;

FIG. 9 is a schematic representation of an electromagnetic field forming over a particle of nano-composite material during operation of the bioreactor; and

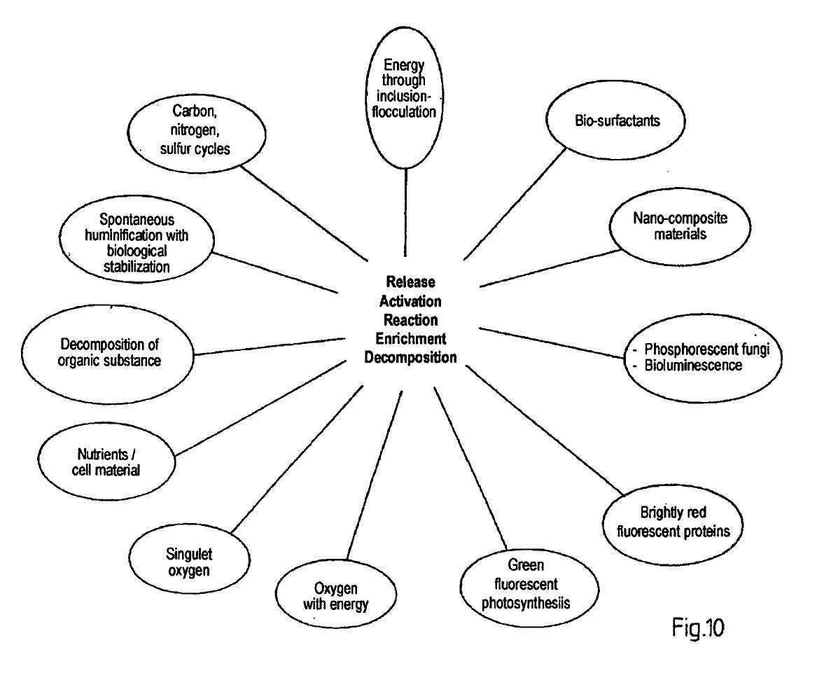

FIG. 10 is a diagram for the evolution of a photodynamic decomposition taking place during use of the bioreactor in accordance with the invention.

FIG. 1 shows a sectional view of a small-scale sewage treatment plant 1 including a mechanical stage which is constituted by a 3-chamber settling tank 4. Such multi-chamber settling tanks may still be found—particular in rural areas—on a large number of premises. In principle this is a matter of a container 6 that is subdivided by a partition wall 8 into three sub-chambers, of which merely a first chamber 10 and another chamber 12 are represented in FIG. 1. The effluent to be purified flows to the 3-chamber settling tank through an inlet 14 to enter into a first chamber (not shown) and may flow off through passages 16 in the walls 8 into the next sub-chamber 12 and from there into the last sub-chamber 10. Substances capable of settling in the single chambers 10, 12 settle by sedimentation, whereas float substances float on the liquid surface 18. The outlet 20 is selected such that the sediments and the float substances remain inside the chambers 10, 12, and the purified effluent is discharged without these pollutants.

For a biological processing, the bioreactor 2 is provided in the chamber 10 as a retrofit kit constituting a biological stage. The main component of this bioreactor is a container or strainer basket 22 which has in the represented practical example the form of a float, i.e., it has sufficient buoyancy for floating in the effluent to be treated biologically. For positioning of the strainer basket 22, a vertical guide 24 is arranged in the chamber 10 which may, for instance, be supported on the partition wall 8 and/or the side walls of the 3-chamber settling tank 6 (see dashed lines in FIG. 1). The strainer basket 22 is arranged to be slidable along this vertical guide 24 in the direction X in FIG. 1, so that it may be moved up or down inside the chamber 10 as a float in accordance with a fluid level 18.

Into the strainer basket 22 catalytically active surfaces provided whereby a particular microbiotic mixture forms a biofilm. This microbiotic mixture consists in the represented practical example of a proportion of photosynthetically active microorganisms and a proportion of light-emitting microorganisms. The interaction between the photosynthetically active microorganisms and the luminous bacteria has the result that the photosynthetically active microorganisms are stimulated to photosynthesis by the emitted light. The microorganisms entertain photosynthesis with hydrogen sulfide and water as the educt while releasing sulfur and oxygen, respectively. They are moreover capable of binding nitrogen as well as phosphate and decomposing organic and inorganic matter. With regard to the concrete composition of this microbiotic mixed culture, reference is made to the same applicant's patent applications DE 100 62 812 A1 and DE 101 49 447 A1 for the sake of simplicity. Having referred to these applications, following the description of the practical examples only the essential steps of this photodynamic decomposition shall be explained.

Interaction of the microbiotic mixture and the catalytic surfaces of the strainer basket 22 results in a photodynamic decomposition of organic substances. This photodynamic decomposition of substances is described, e.g., in the application DE 102 53 334 to the same applicant.

The structure of the strainer basket 22 shall in the following be explained by referring to FIGS. 2 and 3.

In the practical example represented in these figures, the strainer basket 22 has in the lateral view (FIG. 1) an approximately funnel-shaped geometry, so that the diameter of the strainer basket 22 is conically tapered in a downward direction away from the liquid surface 18. The side walls of the strainer basket 22 are in the represented practical example made of stainless steel and may at least partially be provided with a photocatalytically active coating. This coating may—as is indicated in FIG. 2 by the dash-dotted and double-dotted lines—be formed on the inner circumferential wall of the strainer basket 22 and/or on the outer circumferential wall. In the represented practical example, the strainer basket 22 is made of V4A and provided with a titanium dioxide coating. Instead of this titanium dioxide it is also possible to use indium-tin oxide or the like. The outer circumferential wall of the strainer basket 22 is provided with a multiplicity of recesses 26, so that the effluent to be stabilized biologically may enter from the chamber 10 into the strainer basket 22. The lower end face 28 of the strainer basket is closed, so that the flow into the strainer basket 22 substantially takes place in a radial direction. The upper end face may also be closed. In a case where this upper surface is situated above the fluid level, closing is not necessary. Inside the cavity of the strainer basket 22 an exchangeable filler body 30 is received which has a spiral-shaped structure in the top view (FIG. 3). In the represented practical example, this filler body 30 consists of a carrier material which may, e.g., be a spirally, helical stainless steel plate. This spiral shape is adapted to the funnel-shaped structure of the strainer basket 22, i.e. the diameter of the spiral increases in the axial direction from the bottom to the top. The spiral thus lies in the shape of a helical line inside the funnel, with its diameter increasing upwardly in the manner of a cyclone.

On this screw-type helical carrier of stainless steel there is applied on both sides a foam material, e.g., a PU foam coated or admixed with activated charcoal and optionally with nano-composite material. The PU foam results in the formation of a pore system, the walls of which are coated with activated charcoal, so that a large substance exchange surface is provided.

This pore system coated with activated charcoal and with the nano-composite particles forms a comparatively large growth surface for the formation of a biofilm in which the above described mechanisms unfold.

In a further development of the invention, one side of the spiral-shaped filler body 30 is provided with the above mentioned activated charcoal coating, while the other side is additionally coated with a photocatalytically active surface, for example of titanium oxide, which is applied on the activated charcoal layer or on the porous material (e.g., foam material). With the aid of the latter photocatalytically active layer, the above described photodynamic process is accelerated, however by these photocatalytic surfaces the formation of a biofilm is impeded, so that the latter forms on the surface occupied only by activated charcoal. In principle it may also be provided to apply the photocatalytically active layer and the growth surface (activated charcoal) partially, i.e., only in particular wall areas, in a side-to-side arrangement.

Instead of the construction having a central carrier and a coating on either side it is also possible to use a porous body (foam) which by itself only has an insufficient strength. In order to improve the strength of the filler body, this core is then introduced between a double wall of a carrier which in turn may be manufactured of stainless steel or some other suitable material, e.g., acid-resistant plastics, etc.

The microorganisms mentioned at the outset may either be introduced centrally through the intermediary of an apportioning hose into the center of the spiral-shaped filler body 30. It is, however, also possible to introduce these microorganisms into the pore system together with the nano-composite materials already during manufacture of the filler body. Trials have been very promising in which the microorganisms and nano-composite materials were dissolved in quitosane, and this mixture with an addition of the nano-composite materials is then applied on the filler body, e.g., by impregnation, so that a continuous supply of microorganisms is omitted, and it is merely necessary to replace the filler body 30 in regular intervals.

The strainer basket 22 is rotatably fastened at the vertical guide 24 via bearings 34. In principle it is also possible only mount the filler body 30 in a rotatable manner, while the strainer basket 22—or more accurately its jacket—is fixedly attached to the vertical guide 24, so that the filler body 30 is rotatable relative to the jacket.

The temperature increase and a formation of gas during the biological decomposition process described at the outset, and particularly the formation of an alternating electrical field inside the strainer basket 22, bring about a rotation of the strainer basket 22 or of the filler body 30, whereby on the one hand the thorough mixing of the effluent to be treated inside the strainer basket 22, and on the other hand the flow through the strainer basket 22 is improved, with the filler body 30 having a screw-type wavy configuration supporting the flow of effluent.

The above mentioned alternating electrical field is generated during photodynamic processes and is supported by the photocatalytically active coating 32 of the strainer basket 22 as well as by the introduction of the nanostructures, the function of which shall be explained later on by referring to FIG. 9. If the energy introduced from the biological decomposition process is not sufficient to make the filler body 30 or the strainer basket 22 rotate, the latter may also be associated with a separate drive mechanism for application of a torque so as to bring about the rotation.

FIG. 4 shows another practical example of a strainer basket 22 of a bioreactor 2 which has, as a difference from the above described practical example, not a funnel shape but a cylindrical shape.

The jacket 36 of the strainer basket 22 is again provided on both sides or on one side with a photocatalytically active coating (titanium dioxide, indium-tin oxide). Inside this cylindrical jacket 36 there is again arranged a screw-type helical filler body 30 which is formed by a carrier having a pore structure, which is coated with a catalytic surface, for example with activated charcoal. Like in the above described practical example, it is again possible to apply partially or on particular wall portions of the filler body 30 a photocatalytically active surface of titanium dioxide, indium-tin oxide.

Specifically, in the represented practical example the carrier is in turn executed as a sandwich construction. The actual carrier material consists of a VA grid body having a thickness of two to three millimeters, wherein the helical structure is formed by two grid surfaces wherebetween—like in the above described practical example—a semi-hard, open-cell PU foam with an activated charcoal coating is introduced. The grid bars arranged on the downwardly facing side of the helix are provided with a photocatalytic surface, with the mesh size at these downwardly facing major surfaces amounting to approx. 10-12 mm. On the grid bars forming the upwardly facing major surface of the helix no coating is provided. The mesh size here is approx. 25 to 30 mm.

On the downwardly facing side of the helix, the PU foam is coated with a gel-type material of quitosane. In this quitosane the nano-composite materials are embedded which respectively constitute a piezoelectric ceramic system of PZT short fibers with photocatalytic coatings. Moreover microorganisms having a function typical for purification plants and a bio-physical function are jointly embedded. On the top side of the PU foam core in the cationically active quitosane lactate only aerobic microorganisms are installed.

As was already described at the outset, formation of a biofilm occurs very rapidly on the top side of the spiral, with the formation of a biofilm on the bottom side of the sandwich body being prevented by the photocatalytic activities accompanied by a more intense formation of gas (hydrogen and oxygen). The inner and outer sides of the cylindrical strainer basket 22 are in turn—like in the above described practical example—provided with a permanent photocatalytic surface.

In this practical example, too, the external diameter of the helical filler body 30 increases from below in an upward direction. Other than in the above described practical example, in the strainer basket 22 represented in FIG. 4 the lower end face is provided as an entrance cross-section for the effluent to be treated—the peripheral jacket 36 is impermeable to water, so that the flow towards the strainer basket 22 does not take place radially like in the practical example described at the outset, but axially.

Preliminary trials showed that the PU foam of the filler body 30 already sufficiently provides the strainer basket 22 with buoyancy. Where this buoyancy should not be enough, it is possible—in accordance with the indication in FIG. 4—to provide in the upper range of the strainer basket 22 a float element 38 annularly encompassing the cylindrical jacket 36.

Instead of the PU foam coated with activated charcoal it is also possible to use ceramic material having a sufficient pore volume.

The advantage of the practical example represented in FIG. 4 resides in the substantially more simple manufacturing suitability of the jacket 36 and in the lower pressure loss to be expected in the case of an axial through flow.

In the following, another practical example of a bioreactor 2 shall be explained by referring to FIGS. 5 to 8.

In this practical example, the bioreactor 2 is formed to be cylindrical and has a cylindrical strainer basket 22 open at its end face which is in this practical example manufactured of a perforated metal plate, preferably of stainless steel. Instead of a jacket provided with recesses it is also possible to use a closed peripheral jacket which is only open at its end faces. The tube-type strainer basket 22 has, e.g., a length of about 110 cm and a diameter of 35 cm. The preferably circular recesses 26 formed in the tube jacket have in the represented practical example a diameter of about 8 mm and a center distance of 12 mm.

The strainer basket 22 encompasses the helically formed filler body 30 which is in the represented practical example executed with a uniform external diameter, wherein the internal diameter of the strainer basket 22 is executed only slightly larger than the external diameter D of the helix of the filler body 30.

In the represented practical example, the filler body 30 consists of a supporting body 40 formed substantially of a steel tube 42 arranged coaxial with the strainer basket 22, and of round bars 44 spirally arranged thereon. These round bars 44 carry a spiral-shaped mat 46 of PUR foam. The round bars 44 are arranged at right angles with the steel pipe axis 42 and reach barely to the perforated circumferential wall of the strainer basket 22. The PUR mat 46 is—in accordance with the representation in FIG. 6—arranged underneath the round bars 46, so that it is supported in the direction of through flow (from below upwards in FIG. 6).

In the represented practical example, the strainer basket 26 has a standing position, with the filler body 30 being rotatably mounted therein.

Similar to the above described practical example, the PUR mat 46 is provided with a catalytically active layer, preferably an activated charcoal coating. The lower major surface of the mat 46 facing away from the round bars 46 is additionally coated with a biopolymer, e.g. a lactic acid polymer (PLA). In this biopolymer the microorganisms described at the outset and the nano-composite materials are arranged. In addition to, or instead of the PLA, sugar-molasses or quitosane-lactate may also be employed as a carrier material. The microbiotic mixture in accordance with the invention moreover contains micronutrients such as, e.g., aluminum, calcium, cobalt, copper, iron, magnesium, manganese, molybdenum, potassium, nickel, selenium, sulfur, zinc and/or chromium.

The microbiotic mixture may moreover contain microorganisms typical for purification plants.

As was already described, formation of a biofilm takes place very rapidly on the upper side of the spiral-shaped filler body 30, with the formation of a biofilm on the lower side of the mat being prevented by catalytic activities accompanied by an intense formation of gas (hydrogen or oxygen).

The photodynamic decomposition of the organic constituents is moreover supported by the photocatalytic coating of the strainer basket 22. As is in particular visible in the enlarged representation in accordance with FIG. 7, the strainer basket is coated both at its inner circumferential surface and on its outer circumferential surface with a photocatalytically active layer, e.g., titanium dioxide. This layer is applied fully on the inner circumferential surface, i.e. at the side facing the filler body 30, whereas on the outer circumferential surface in accordance with FIGS. 5 and 7 the titanium dioxide is applied in the form of stripes 48 between which uncoated areas 50 remain. These coated and uncoated areas 48, 50 extend in the longitudinal direction of the strainer basket 22. In the represented practical example, the width of the stripes 48 about corresponds to the spacing of four hole-type recesses 26, whereas the width of the uncoated areas 50 is substantially smaller and about corresponds to the spacing between two adjacent recesses 26.

In cooperation with the catalytic coating of the strainer basket 22 and the above described coating of the helical filler body 30 a comparatively strong electromagnetic field manifests above the bioreactor and allows to tap a voltage or use it for a rotational drive of the filler body 30 inside the strainer basket 22 or of the entire strainer basket 22.

Another particularity of the bioreactor 2 is represented in FIG. 8. Accordingly, the circular recesses 26 are in the represented practical example preferably formed by punching, with a punching burr 52 protruding to the inside, i.e., towards the filler body 30. The above described photocatalytically active coating 32 of titanium-dioxide is in this practical example applied following blanking of the recesses 26. It was found that the coating frequently will not adhere in the range of the extremely sharp-edged punching burrs 52, so that these burrs 52 remain uncoated. Surprisingly, preferably a biofilm 54 adheres at these uncoated punching burrs 52 during operation of the bioreactor 2—i.e., these uncoated areas thus act as germination zones for the formation of the biofilm on the inner circumferential surface of the reactor, so that the conversion of the organic constituents is improved further.

The mechanisms underlying the formation of the electromagnetic field shall be explained by referring to the schematic representation in FIG. 9.

FIG. 9 shows in a strongly diagrammatic form an elongated nanoparticle produced from PZT fibers (lead circonate—lead titanate). This piezoelectric fiber material is initially polarized in a d.c. field in the represented direction of arrow. The long fiber is subsequently provided with a titanium dioxide layer, with such coating being carried out, e.g., by immersion and evacuation of excess material. Drying is carried out at 450° C., wherein the titanium dioxide layer is transformed into a photocatalytically active anatas phase.

Following this coating process, the single particles are cut in the electromagnetic alternating field, so that the end faces 58 are again uncoated. These uncoated areas are in a subsequent manufacturing step—such as by sputtering—provided with aluminum or the like, so that the nanoparticle 56 consists in the completed condition of end-side pole caps, a titanium dioxide coating, and a piezoelectric core.

During operation of the bioreactor, the pole ends 60, 62 formed by the aluminum caps are ionized by deposition of cations (left side in FIG. 9) and anions (right side in FIG. 9) as metabolic products of the microorganisms. This ionization of the pole ends 60, 62 results in the development of a relatively strong electromagnetic field, the field lines 64 of which are represented in FIG. 9.

Due to the comparatively small surface area of the pole ends 60, 62 it is possible to observe a strong increase of the field strength at these pole ends 60, 62. This electric point effect results in the range of the pole ends 60, 62 in a collision ionization of the gas molecules due to already existing charge carriers that are strongly accelerated in the vicinity of these pole ends 60, 62. Concurrently with this discharge an “electric wind” is generated which blows away from the two pole ends 60, 62: the nanoparticle 46 thus acts in the manner of a “photon pump” whereby photons are emitted spontaneously, resulting in the creation of blue light beams 64 and red light beams 66 at these pole ends 60, 62.

In accordance with the schematic representation in FIG. 11, an inclusion flocculation of the organic constituents occurs in a first step of the photodynamic decomposition, with energy being released during this inclusion flocculation.

In order to overcome boundary surfaces between the organic constituents and the effluent, bio-surfactants (bile acid) are produced by the microorganisms and result in contact surface acidification. These bio-surfactants are surface-active substances produced by the microorganisms which have a stabilizing effect and allow the bacteria to enter into contact with the contaminants and dissolve them. A contact surface acidification brings about an increase in boundary surface conductivity. At the boundary surface between flake and fluid there occurs, owing to isomorphous exchange of lattice atoms, the formation of negative surface charges, bringing about a deposition of cations of the electrolyte (Stern layer). In the subsequent layer the diffusion of ions results in a gradual reduction of the cation concentration and increase of the anion concentration.

Nano-composite materials are added to the microbiotic mixture as further constituents. This is a piezoelectric ceramic system of PZT short fibers having a length of 20 to 50 mm. These short fibers are coated photocatalytically, with titanium dioxide or indium-tin oxide being used as a coating material. The natural oscillation of these elements at 50 to 500 kHz results in phosphorescence, a form of luminescence wherein other than in the case of fluorescence, the emission of light takes place with a temporal delay. As a result of this stimulation, energy in the form of radiation having mostly greater wavelengths (354 to 450 nm) is emitted.

The released vibrational energy results in phosphorescence of fungi through stimulation and in the biocatalytic reaction of the bioluminescence of bacteria (vibrio fischeri). This bioluminescence results in a release of fluorescent protein (sea Anemone® anemonia sulcata) which has a bright red fluorescence (633 nm) under blue light.

The microorganisms release color pigments, for instance Monascus pururus, Limicola-Nadson (cell dye 2145) and Pseudomonas fluorescens. With the aid of the bacteriochlorophyll (cyanobacteria) there results the chlorophyll A reaction with an intense green fluorescence at 684 nm. Owing to interaction with cold blue light there results an electron transfer in the purple bacterium and a release of oxygen. Due to the porphyrin synthesis of the cyanobacteria in combination with microalgae of the species (Chlorella vulgaris) and quitosane-lactate as well as due to the absorption of cold blue light (469 to 505 nm), PpIX is charged similarly to a small battery and may thus transfer part of the energy to normal oxygen. These “bio-fuel-cells” moreover make use of the sugar metabolism by transferring electrons from the sugar to the oxygen metabolism with the aid of biocatalysts.

Parallel with the energetic enrichment of the oxygen formed by photosynthesis, reactive singlet oxygen is released.

This “non-mechanical cell digestion process” increasingly releases organic material and affords a very high degree of digestion at a clearly lower introduction of energy, particularly with gram-positive bacteria.

The partial mineralization takes place due to the completely anoxic decomposition of the organic substances in a voltage field of 1200 to 1500 mV. This voltage field is established between the bright red fluorescent light (633 nm) and the green chlorophyll fluorescence (634 nm).

During mineralization a spontaneous huminification occurs in which the pollutants and their metabolites are stabilized biologically and may then not be re-immobilized again.

Finally there occurs a complete mineralization by microorganisms into mineral (inorganic) chemical compounds. As a result, the carbon primarily fixed through photosynthesis in biomass is again freed in the form of carbon dioxide (carbon cycle), and the organically bound nitrogen, sulfur, and the phosphate are split off as an oxidized or reduced inorganic compound (nitrogen cycle, sulfur cycle), so that they are again available to the environment as nutrients (mineral substances, nutrient salts).

By means of the biological stage in accordance with the invention it is possible to reduce the organic proportion of the dry substance (TS) in the strainer basket (bioreactor) to less than 10% of the dry substance owing to decomposition of the inhibiting substance and the release of oxygen and energy. The reactive singlet oxygen released by the energy enrichment of the oxygen does, for instance, most effectively oxidize hormonal residues and antibiotics. After a few seconds, organic substances are converted by disintegration and are subsequently rendered innocuous. The biofilm at the upper side of the helical insert, on the other hand, decomposes the substances dissolved by effluent.

What is disclosed is a bioreactor comprising a strainer basket, inside which a filler body consisting of a porous carrier having a high specific surface area is received. Into this strainer basket a mixture of microorganisms is introduced which preferably includes a proportion of photosynthetically active microorganisms and a proportion of light-emitting microorganisms, so that a photodynamic decomposition of organic substances takes place. In accordance with the invention, the mixture of microorganisms contains a proportion of photocatalytically active nanoparticles.

### LIST OF REFERENCE NUMERALS

1 small-scale sewage treatment plant
2 biological stage
4 mechanical stage
6 3-chamber settling tank
8 partition wall
10 chamber
12 chamber
14 inlet
16 recess
18 fluid level
20 outlet
22 strainer basket
24 vertical guide
26 recess
28 end face
30 filler body
32 coating
34 bearing
36 jacket
38 float element
40 supporting body
42 steel tube
44 round bar
46 mat
48 stripe
50 uncoated areas
52 punching burr
54 biofilm
56 nanoparticle
58 end face
60 pole end
62 pole end
64 blue light
66 red light

1. A bioreactor for the treatment of contaminated communal or industrial effluent, or of fluids contaminated with organic pollutants, in particular for a small-scale sewage treatment plant, wherein microorganisms for decomposing organic pollutants are contained, characterized by a container including at least one recess for the passage of the effluent to be treated, inside of which a filler body having a large pore volume as well as a microbiotic mixture, preferably comprising a proportion of photosynthetically active microorganisms and a proportion of light-emitting microorganisms, is provided.

2. The bioreactor in accordance with claim 1, wherein the filler body has a spiral shape.

3. The bioreactor in accordance with claim 2, wherein the diameter of the spiral-shaped filler body axially increases towards the liquid surface.

4. The bioreactor in accordance with claim 1, wherein the filler body comprises a supporting layer on which a foam material is applied.

5. The bioreactor in accordance with claim 1, wherein the filler body has a preferably grid-shaped, double wall wherebetween a foam material is arranged.

6. The bioreactor in accordance with claim 1, wherein the filler body consists of a ceramic material having a large pore volume.

7. The bioreactor in accordance with claim 4, wherein the foam material, preferably PU foam, is provided with a catalytically active surface, for example with activated charcoal or the like.

8. The bioreactor in accordance with claim 7, wherein microorganisms are applied on a surface of the filler body, or the microorganisms are centrally introduced inside the strainer basket.

9. The bioreactor in accordance with claim 8, wherein the microorganisms are received in a carrier substance, e.g., quitosane or a biopolymer, for example lactic acid polymer.

10. The bioreactor in accordance with claim 9, wherein the microbiotic mixture further contains nanoparticles in addition to the microorganisms.

11. The bioreactor in accordance with claim 7, wherein the filler body is provided with the microbiotic mixture on the one hand and with a layer favoring formation of a biofilm, e.g., with activated charcoal, on the other hand.

12. The bioreactor in accordance with claim 2, wherein the container walls and/or surface areas of the filler body are coated with a photocatalytically active layer.

13. The bioreactor in accordance with claim 10, wherein the layer is titanium dioxide or indium-tin oxide.

14. The bioreactor in accordance with claim 12, wherein the photocatalytic layer is applied largely continuously on the inner circumferential surface of the container and in portions on the outer circumferential surface.

15. The bioreactor in accordance with claim 14, wherein the photocatalytic layer on the outer circumferential surface is applied in the form of stripes, wherein these preferably extend in the longitudinal direction.

16. The bioreactor in accordance with claim 1, wherein recesses of the container are punched out, so that punching burrs project inwardly, and the photocatalytic coating is applied following punching.

17. The bioreactor in accordance with claim 1, wherein the container has a cylindrical shape and is provided on the end sides with at least one recess for the passage of liquid.

18. The bioreactor in accordance with claim 1, wherein the container or the filler body is mounted rotatably.

19. Microbiotic mixed culture for the decomposition of organic constituents in fluids, in particular for use in a bioreactor in accordance with any one of the preceding claims, comprising a proportion of photosynthetically active microorganisms and a proportion of light-emitting microorganisms in a biological solution, characterized in that the mixed culture contains a proportion of piezoelectrically active nano-composite materials, the surface of which is provided with a photocatalytically active layer.

20. A mixed culture in accordance with claim 19, wherein the nano-composite material has a fiber-type structure with a length of 20 to 100 mn and a diameter of 2 to 10 nm.

21. The mixed culture in accordance with claim 19, wherein the coating contains titanium dioxide or indium-tin oxide.

22. The mixed culture in accordance with claim 19, wherein the coating of the nano-composite materials is provided with multiple recesses for the formation of pole sites.

23. The mixed culture in accordance with claim 20, wherein the coating of the nano-composite particles is interrupted at the end sides, and a respective pole is formed at the two end sides.

24. A retrofit kit for a small-scale sewage treatment plant, comprising;
a bioreactor for the treatment of contaminated communal or industrial effluent, or of fluids contaminated with organic pollutants, in particular for the small-scale sewage treatment plant, wherein microorganisms for decomposing organic pollutants are contained, characterized by a container including at least one recess for the passage of the effluent to be treated, inside of which a filler body having a large pore volume as well as a microbiotic mixture, preferably comprising a proportion of photosynthetically active microorganisms and a proportion of light-emitting microorganisms, is provided;
and the microbiotic mixed culture in accordance with claim 19.
